(19) United States
(12) Patent Application Publication
(10) Pub. No.: US 2006/0194845 A1
(43) Pub. Date: Aug. 31, 2006

(54) Use of ALK 5 inhibitors to modulate or inhibit myostatin activity leading to increased lean tissue accretion in animals

(75) Inventor: David G. Sawutz, Maplewood, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/190,453

(22) Filed: Jul. 27, 2005

Related U.S. Application Data
(60) Provisional application No. 60/592,359, filed on Jul. 29, 2004.

(51) Int. Cl. A61K 31/4439; A61K 31/4164

(52) U.S. Cl. 514/341; 514/396

(57) ABSTRACT

The present invention provides methods of increasing muscle tissue in animals. In one aspect of the invention, the method includes administering an effective amount of an ALK 5 receptor inhibitor such as 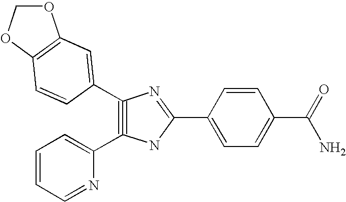 to an animal for a time sufficient to cause the desired effect.

### CROSS-REFERENCE TO RELATED APPLICATIONS

This application is a non-provisional application that claims priority under 35 U.S.C. § 119(e) of provisional application U.S. Ser. No. 60/592,359 filed Jul. 29, 2004, the contents of which are hereby incorporated by reference in their entireties.

### FIELD OF THE INVENTION

The present invention is directed to methods and chemical compositions for increasing lean muscle tissue in non-human animals such as livestock.

### BACKGROUND OF THE INVENTION

Over the years, various methods have been proposed to increase the amount of muscle tissue of animals and improve the ratio of lean to fat deposition. The advantages of animals with such properties as compared to their untreated counterparts are readily discernable and include, for example, lower production costs, improved feed conversion efficiency, healthier livestock, healthier foods obtained therefrom, better product quality, etc.

Many efforts in this regard have centered around the use of livestock feeds which have been enhanced or fortified in some way. Such feeds, however, can be expensive and the gains in muscle tissue for the livestock are somewhat limited or not always evident in actual use. Other attempts to increase the muscle tissue content of livestock animals have focused on the administration of anabolic steroids and/or hormones. While such agents can increase the amount of muscle tissue and often reduce the amount of adipose or fat tissue in animals, consumers have not embraced this technology. In fact, there is significant consumer resistance associated with purchasing meats or foods obtained from animals that have been treated with hormones or steroids. For example, the European Union has banned hormonal growth promoters (HGPs), including bovine growth hormone (GH), porcine and equine GH.

Some efforts related to improving the muscle/fat ratio have focused on the discovery of hormones secreted by muscle and fat cells. These hormones regulate feed intake, energy metabolism, and body composition. Leptin, adiponectin and myostatin were discovered through the study of genetically obese, or double-muscled animals. While it is certainly possible to envision future transgenic livestock species which exploit these findings, it is likely that consumer acceptance of meats obtained from transgenic animals will still be low.

Myostatin, previously known as growth differentiation factor 8 or GDF8, is a type of transforming growth factor, β (TGF-β). It is a potent negative regulator of skeletal muscle growth and a regulator of adipogenisis. Myostatin null mice have been shown to display increases in muscle mass and decreased fat accumulation. Inhibition of myostatin with blocking antibodies increases muscle mass.

TGF-β cytokines signal through a family of single transmembrane serine/threonine kinase receptors. These receptors can be divided in two classes, the type I or activin like kinase (ALK) receptors and type II receptors. A recent publication by Rebbapragada, A. et al. (Molecular and Cellular Biology, Vol. 23, No 20., October 2003, p 7230-7242) suggests that, like TGF-β cytokines, myostatin binds to and activates a Type II receptor complex including ALK4 or ALK5. The ALK receptors are distinguished from the Type II receptors in that the ALK receptors (a) lack the serine/threonine rich intracellular tail, (b) possess serine/threonine kinase domains that are very homologous between Type I receptors, and (c) share a common sequence motif called the GS domain, consisting of a region rich in glycine and serine residues. The GS domain is at the amino terminal end of the intracellular kinase domain and is believed to be critical for activation by the Type II receptor. Several studies have shown that TGF-β signaling requires both the ALK (Type I) and Type II receptors. Specifically, the Type II receptor phosphorylates the GS domain of the Type I receptor for TGF-8 ALK5, in the presence of TGF-β. The ALK5, in turn, phosphorylates the cytoplasmic proteins smad2 and smad3 at two carboxy terminal serines. Generally, it is believed that in many species, the Type II receptors regulate cell proliferation and the Type I receptors regulate matrix production.

Various ALK5 receptor inhibitors have been described. See, for example, U.S. Pat. No. 6,465,493, as well as US Patent Application Publication Nos. US2003/0149277, US2003/0166633, US20040063745, and US2004/0039198, the contents of each of which are incorporated herein by reference. These publications describe inter alia various pyridinylimidazoles and their use in the treatment of ALK5 mediated disease states. There is no disclosure or suggestion about their use in methods of increasing muscle tissue or decreasing fat tissue in animals.

Since ALK5 receptors are not associated with cell proliferation, it was not believed that administering ALK5 receptor inhibitors to animals would have any appreciable effect on the muscle/fat ratio.

There remains a need for proving effective methods for producing livestock with higher proportions of lean muscle and/or lower levels of fat tissue.

The citation of any reference herein should not be construed as an admission that such reference is available as “prior art” to the instant application.

### SUMMARY OF THE INVENTION

The present invention generally relates to methods and compositions for increasing lean muscle tissue in animals such as livestock. In one embodiment, there is provided a method of increasing muscle tissue in animals which includes, administering an effective amount of an activin-like kinase (ALK) 5 receptor inhibitor or an ALK5/ALK4 dual inhibitor to an animal in which an increase in muscle mass is desirable.

In a particular embodiment, a composition comprises an inhibitor for the ALK5 receptor. In one aspect of this embodiment, the composition comprises an inhibitor that specifically inhibits the ALK5 receptor and the ALK4 receptor. In a particular embodiment of this type, the composition comprises an inhibitor that is specific for inhibiting the ALK 5 receptor.

In preferred aspects of this embodiment, the ALK 5 receptor inhibitor is

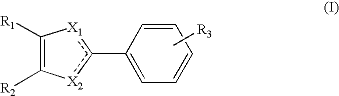

wherein R₁ is H, naphthyl or phenyl optionally substituted with one or more substituents selected from among halo, —O—C₁₋₆ alkyl, —S—C₁₋₆ alkyl, C₁₋₆ alkyl, C₁₋₆ haloalkyl, —O—(CH₂)ₙ₁—Ph, —S—(CH₂)ₙ₁—Ph, cyano, phenyl, and CO₂R₄, wherein R₄ is hydrogen or C₁₋₆ alkyl, and n1 is 0, 1, 2 or 3; or R₁ is phenyl fused with an aromatic or non-aromatic cyclic ring of 5-7 members wherein the cyclic ring optionally contains up to two heteroatoms, independently selected from N, O and S;

R₂ is H or 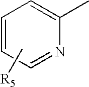
wherein R₅ is H, C₁₋₆ alkyl, C₁₋₆ alkoxy, phenyl, NH(CH₂)ₙ₂—Ph or NH—C₁₋₆ alkyl, or halo, wherein n2 is 0, 1, 2 or 3;

R₃ is CONR₆R₇, CN, NO₂, C₁₋₆ alkylthio, —SO₂—, C₁₋₆ alkyl, C₁₋₆ alkoxy, SONH₂, CONHOH, NH₂, CHO, CH₂OH, CO₂R₆, tetrazole, OH, —S—C₁₋₆ alkyl, —SO—C₁₋₆ alkyl, —O—C₁₋₆ alkyl, (CH₂)ₙ₃NH₂, CONHOR₆, O(CH₂)ₙ₃CO₂R₆, O(CH₂)ₙ₃CONH R₆, CONHR₆, (CH₂)ₙ₃CO₂R₆, or (CH₂)ₙ₃CONHR₆ wherein R₆ and R₇ are independently H or a C₁₋₆ alkyl and n3 is 0, 1, 2 or 3; and

one of X₁ and X₂ is N, S, O or CR₈, and the other is N 8 or CHR₈ wherein R₈ is hydrogen, C₁₋₆ alkyl, or C₃₋₇ cycloalkyl, or when one of X₁ and X₂ is N or CR₈, then the other is S or O.

In more preferred aspects, the ALK5 receptor inhibitor is either:

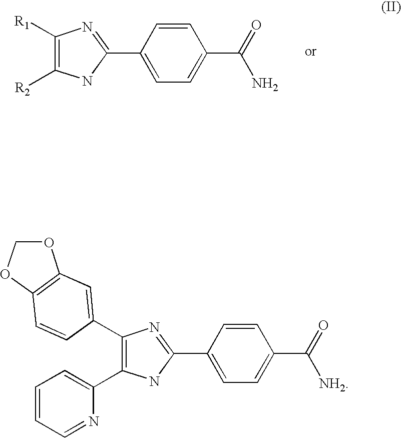

While it is contemplated that the methods of the present invention will useful in the treatment of a wide variety of animals, some preferred ones include ruminants, avian species, fish, swine and livestock animals such as cattle, poultry, pigs, goats and sheep. The amount of the ALK 5 inhibitor administered to the animal will vary, depending on the agent selected and size of animal being treated, but is generally within the range of from about 0.01 to about 100 mg/kg/day.

Further aspects of the invention include those in which the administering of the ALK5 receptor inhibitor results in a decrease in the amount of fat tissue in the animal either in combination with the resulting increase in muscle tissue or substantially apart from the muscle tissue growth observed.

Still further aspects of the invention include pharmaceutical dosage forms and/or livestock feeds containing an effective amount of a composition of an inhibitor described herein as well as a kit for increasing muscle deposition in animals which includes an effective amount of a composition of that inhibitor such as those of Formula (I).

Other than in the operating examples, or where otherwise indicated, all numbers expressing quantities of ingredients, reaction conditions, and so forth used in the specification and claims are to be understood as being modified in all instances by the term “about”.

As a result of the present invention, it has been surprisingly found that it is possible to significantly increase the amount of lean muscle and improve the muscle/fat ratio in animals using ALK5 receptor inhibitors.

### DETAILED DESCRIPTION

In certain embodiments, the present invention is directed to methods of increasing muscle tissue in an animal, and/or decreasing the amount of fat tissue in an animal. The methods are carried out by administering an effective amount of an activin-like kinase (ALK) 5 receptor inhibitor to an animal to which it is desired to have its muscle mass increased. (The animals don't need treatment per se; rather they are being treated to increase performance as measured by increased lean tissue) Though, the present invention is not bound by any particular theory, it is suggested that the desirable effects observed when ALK5 and/or ALK4 receptor inhibitors are administered to animals, the increase in muscle mass is due, at least in part, to inhibition of the Ser/Thr kinase activity associated with ALK5.

Some preferred ALK 5 receptor inhibitors useful in the practice of the invention correspond to formula (I) 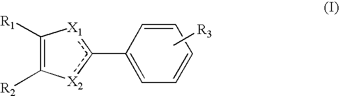
wherein R₁ is H, naphthyl or phenyl optionally substituted with one or more substituents selected from among halo, —O—C₁— alkyl, —S—C₁₋₆ alkyl, C₁₋₆ alkyl, C₁₋₆ haloalkyl, —O—(CH₂)ₙ₁—Ph, —S—(CH₂)ₙ₁—Ph, cyano, phenyl, and CO₂R₄, wherein R₄ is hydrogen or Cue alkyl and n1 is 0, 1, 2 or 3; or R₁ is phenyl fused with an aromatic or non-aromatic cyclic ring of 5-7 members, wherein the cyclic ring optionally contains up to two heteroatoms, independently selected from N, O and S;

R₂ is H, or

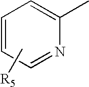

wherein R₅ is H, Cue alkyl, Con alkoxy, phenyl, NH(CH₂)ₙ₂-Ph or NH—C₁— alkyl, or halo, wherein n2 is 0, 1, 2 or 3;

R₃ is CONR₆R₇, CN, NO₂, C₁₋₆ alkylthio, —SO₂—, C₁₋₆ alkyl, C₁₋₆ alkoxy, SONH₂, CONHOH, NH₂, CHO, CH₂OH, CO₂R, tetrazole, OH, —S—C₁₋₆ alkyl, —SO—C₁₋₆ alkyl, —O—C₁— alkyl, (CH₂)ₙ₃NH₂, CONHOR₆, O(CH₂)ₙ₃CO₂R₆, O(CH₂)ₙ₃CONHR₆, CONHR₆, (CH₂)ₙ₃CO₂R₆, or (CH₂)ₙ₃CONHR₆, wherein R₅ and R₇ are independently H or a C₁₋₆ alkyl, and n3 is 0, 1, 2 or 3; and

one of X₁ and X₂ is N, O, S or CR₈, and the other is NR₈ or CHR₈,

wherein R₈ is hydrogen, OH, C₁₋₆ alkyl, or C₃₋₇ cycloalkyl. Or when one of X₁ and X₂ is N or CR₈, then the other is S or O.

Pharmaceutically acceptable salts or solvates thereof are also contemplated.

Within the scope of formula (I), some more preferred ALK 5 receptor inhibitors include: 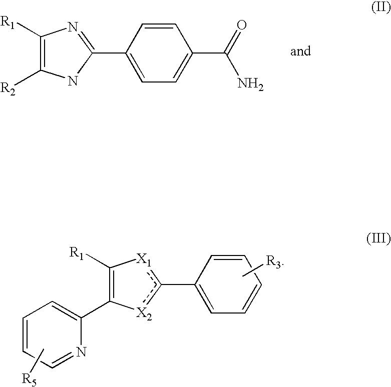
wherein all variables are as previously defined.

A compound for use in the present invention, exemplified below is:

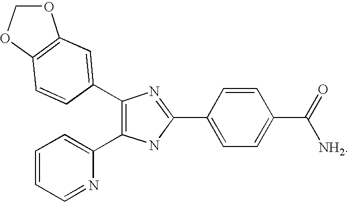

As used herein, the double bond indicated by the dotted lines of formulas (I) and (III), represent the possible tautomeric ring forms of the compounds falling within the scope of this invention. It will be understood that when one of X, and X₂ is carbon and the other is nitrogen, then the double bond could be either to the carbon or the nitrogen. When X₁ and X₂ are both carbon, then the double bond could be to either X₁ or X₂, or to between X₁ and X₂. When X₁ and X₂ are both nitrogen, then the double bond is to the unsubstituted nitrogen.

Preferably R₁ is an optionally substituted naphthyl or phenyl. More preferably R₁ is phenyl optionally substituted with one or more substituents selected from among halo, C₁₋₆ alkoxy, C₁₋₆ alkylthio, and phenyl. Alternatively R₁ can be phenyl fused with an aromatic or non-aromatic cyclic ring of 5-7 members wherein the cyclic ring optionally contains up to two heteroatoms, independently selected from N, O and S, and is optionally substituted by ═O. Examples of R₁ include benzo[1,3]dioxolyl, 2,3-dihydrobenzo[1,4]dioxinyl, benzoxazolyl, benzothiazolyl, quinoxalinyl, benzo[1,2,5]oxadiazolyl, benzo[1,2,5]thiadiazolyl, [1,2,4]triazdo[1,5a]pyridyl-dihydrobenzofuranyl, benzo[1,4]oxazinyl-3-one or benzoxazolyl-2-one.

Preferably, when R₅ is not H, R₅ is positioned ortho to the nitrogen of the pyridyl ring. In a particular embodiment, R₅ is methyl. Preferably R₃ is CO₂H, CONH₂, CN, CONHOH, CH₂OH or tetrazole.

Preferably one of X₁ and X₂ is N or CR₈, and the other is NR₈ or CHR₈ wherein R₈ is hydrogen, C₁₋₆ alkyl, or C₃₋₇ cycloalkyl, provided that at least one of X₁ and X₂ is N or NR; or one of X₁ and X₂ is N and the other is O. More preferably one of X₁ and X₂ is N and the other is NR₈. Preferably each R₈ is hydrogen.

Some additional compounds which can be used in the methods of the present invention include:
4-[4-(4-Fluorophenyl-5-(2-pyridyl)-1-hydroxy-1H-imidazol-2-yl]-benzonitrile;
4-[4-(4-Fluorophenyl-5-(2-pyridyl)-1H-imidazol-2-yl]-benzonitrile;
4-[4-(4-Fluorophenyl)-5-(2-pyridyl)-1H-imidazol-2-yl]-benzoic acid;
Methyl 4-[4-(4-fluorophenyl)-5-(2-pyridyl)-1H-imidazol-2-yl]-benzoate;
Ethyl 4-[4-(4-fluorophenyl)-5-(2-pyridyl)-1H-imidazol-2-yl]-benzoate;
4-(4-Benzo[1,3]dioxol-5-yl-1-hydroxy-5-pyridin-2-yl-1H-imidazol-2-yl)-benzonitrile;
4-(4-Benzo[1,3]dioxol-5-yl-5-pyridin-2-yl-1H-imidazol-2-yl)-benzonitrile;
4-(4-Benzo[1,3]dioxol-5-yl-5-pyridin-2-yl-1H-imidazol-2-yl)-benzoic acid;
2-[4-Benzo[1,3]dioxol-5-yl-2-(4-nitrophenyl)-1H-imidazol-5-yl]-pyridine;
3-(4-Benzo[1,3]dioxol-5-yl-5-pyridin-2-yl-1H-imidazol-2-yl)-phenylamine;
4-[4-(4-Fluorophenyl)-2-(4-nitrophenyl)-1H-imidazol-5-yl]-pyridine;
4-[4-(4-Fluorophenyl)-5-pyridin-2-yl-1H-imidazol-2-yl)phenylamine;
4-(4-Benzo[1,3]dioxol-5-yl-5-pyridin-2-yl-1H-imidazol-2-yl)-phenyl]methanol;
4-(4-Benzo[1,3]dioxol-5-yl-5-pyridin-2-yl-1H-imidazol-2-yl)-benzamide;
4-[4-(2,3-Dihydro-benzo[1,4]dioxin-6-yl)-5-pyridin-2-yl-1H-imidazol-2-yl]-benzonitrile;
4-[4-(2,3-Dihydro-benzo[1,4]dioxin-6-yl)-5-pyridin-2-yl-1H-imidazol-2-yl]-benzamide;
4-[4-(2,3-Dihydro-benzofuran-5-yl)-5-pyridin-2-yl-1H-imidazol-2-yl]-benzamide;
3-[4-Benzo[1,3]dioxol-5-yl-5-pyridin-2-yl-1H-imidazol-2-yl)-benzonitrile;
4-[4-(2,3-Dihydro-benzofuran-6-yl)-5-pyridin-2-yl-1H-imidazol-2-yl]-benzonitrile;
4-[4-(2,3-Dihydro-benzofuran-6-yl)-5-pyridin-2-yl-1H-imidazol-2-yl]-benzamide;
3-(4-Benzo[1,3]dioxol-5-yl-5-pyridin-2-yl-1H-imidazol-2-yl)-benzoic acid;
4-[4-(4-Methoxyphenyl)-5-(2-pyridyl)-1H-imidazol-2yl]-benzonitrile;
4-[4-(2,2-Difluoro-benzo[1,3]dioxol-5-yl)-5-pyridin-2-yl-1H-imidazol-2-yl]-benzamide;
4-[4-(2,3-Dihydro-benzo[1,4]dioxin-6-yl)-1-methyl-5-pyridin-2-yl-1H-imidazol-2-yl]-benzamide;
4-[5-(2,3-Dihydro-benzo[1,4]dioxin-6-yl)-1-methyl-4-pyridin-2-yl-1H-imidazol-2-yl]-benzamide;
4-(5-Benzo[1,3]dioxol-5-yl-4-pyridin-2-yl-oxazol-2-yl)-benzonitrile;
4-(5-Benzo[1,3]dioxol-5-yl-4-pyridin-2-yl-oxazol-2-yl)-benzamide; and
4-(4-Benzo[1,3]dioxol-5-yl-5-pyridin-2-yl-1H-pyrrol-2-yl)-benzamide; or
   a pharmaceutically acceptable salt and/or solvate thereof.

In alternative aspects of the invention, compounds useful in the practice of the invention include the following:
4-(4-Benzo[1,3]dioxol-5-yl-5-pyridin-2-yl-1H-imidazol-2-yl)-phenol;
4-(4-Benzo[1,3]dioxol-5-yl-5-pyridin-2-yl-1H-imidazol-2-yl)-N-methy-1-benzamide;
4-(4-Benzo[1,3]dioxol-5-yl-5-pyridin-2-yl-1H-imidazol-2-yl)-N-methoxy-benzamide;
2-{4-Benzo[1,3]dioxol-5-yl-2-[4-(2H-tetrazol-5-yl)-phenyl]-1H-imidazol-5-yl}-pyridine;
[4-(4-Benzo[1,3]dioxol-5-yl-5-pyridin-2-yl-1H-imidazol-2-yl)-phenoxy]-acetic acid;
4-[4-(4-Fluoro-3-methoxyphenyl)-5-(6-methylpyridin-2-yl)-1H-imidazo-l-2-yl]-benzonitrile;
4-[4-(4-Fluoro-3-methoxyphenyl)-5-(6-methylpyridin-2-yl)-1H-imidazo-l-2-yl]-benzamide;
4-[4-(3-Fluoro-4-methoxyphenyl)-5-(6-methylpyridin-2-yl)-1H-imidazo-l-2-yl]-benzonitrile;
4-[4-(3-Fluoro-4-methoxyphenyl)-5-(6-methylpyridin-2-yl)-1H-imidaz-ol-2-yl]-benizamide;
4-[4-Benzo[1,2,5]oxadiazol-5-yl-5-(6-methyl-pyridin-2-yl)-1H-imidazol-2-yl]-benzonitrile;
4-[4-Benzo[1,2,5]oxadiazol-5-yl-5-(6-methylpyridin-2-yl)-1H-imidazo-l-2-yl]-benzamide;
4-[4-(6-Methoxynaphthalen-2-yl)-5-(6-methylpyridin-2-yl)-1H-imidazo-l-2-yl]-benzonitrile;
4-[4-(6-Methoxynaphthalen-2-yl)-5-(6-methylpyridin-2-yl)-1H-imidazo-l-2-yl]-benzamide;
4-[4-Benzo[1,2,5]thiadiazol-5-yl-5-(6-methylpyridin-2-yl)-1H-imidaz-ol-2-yl]-benzonitrile;
4-[4-Benzo[1,2,5]thiadiazol-5-yl-5-(6-methylpyridin-2-yl)-1H-imidaz-ol-2-yl]-benzamide
4-[4-Benzo[1,3]dioxol-5-yl-5-(6-methylpyridin-2-yl-1H-imidazol-2-yl-)benzonitrile;
4-[4-Benzo[1,3]dioxol-5-yl-5-(6-methylpyridin-2-yl)-1H-imidazol-2-yl-]benzamide;
6-[2-(4-Cyanophenyl)-5-(6-methylpyridin-2-yl)-1H-imidazole-4-yl]-quinoxaline; and
6-[2-(4-Carboxamidophenyl)-5-(6-methylpyridin-2-yl)-1H-imidazole-4-yl]-quinoxaline;

and pharmaceutically acceptable salts and/or solvates thereof.

Synthesis of compounds corresponding to Formulas I-III and the specific molecules identified herein is described, for example, in the aforementioned U.S. Pat. No. 6,465,493, as well as US Patent Application Publication Nos. US2003/0149277, US2003/0166633, US20040063745, and US2004/0039198. Synthesis of the compounds will also be apparent to those of ordinary skill and does not require undue experimentation.

In one preferred embodiment, the animal is a “food-producing” animal, and the result of the administration of the ALK 5 receptor inhibitor is a gain in animal weight, particularly muscle mass, and/or decrease in fat tissue relative to animals not treated with the ALK 5 receptor inhibitor.

For purposes of the present invention, the animals which are preferably treated in accordance with the present invention are food producing animals. The term “food-producing” animal shall be understood to include all livestock animals bred for consumption, e.g., by humans or other animals. A non-limiting list of such animals include those of the avian, ruminants such as bovine, ovine, deer, etc., families, ungulates, as well as aquatic animals, including fish such as trout or salmon, and other species raised or harvested for human consumption. Avian species shall be understood to include, for example, chickens, turkeys, geese, duck, etc. Bovine shall be understood to include, for example, cattle, beef, veal, etc. Ovine shall be understood to include, for example, sheep, etc. Swine or porcine family members are also contemplated.

For purposes of the present invention, the term “fish” shall be understood to include without limitation, the Teleosti grouping of fish, i.e., teleosts. Both the Salmoniformes order (which includes the Salmonidae family) and the Perciformes order (which includes the Centrarchidae family) are contained within the Teleosti grouping.

Examples of potential fish recipients include the Salmonidae family, the Serranidae family, the Sparidae family, the Cichlidae family, the Centrarchidae family, the three-Line Grunt (Parapristipoma ttilineatum), and the Blue-Eyed Plecostomus (Plecostomus spp).

| | |
|---|---|
| TAXON NAME | COMMON NAME |

Salmonidae Family

| | |
|---|---|
| Coregonus clupeaformis | Lake whitefish |
| Coregonus hoyi | Bloater |
| Oncorhynchus keta | Chum salmon |
| Oncorhynchus gorbuscha | Pink salmon |
| Oncorhynchus kisutch | Coho salmon |
| | (silver salmon) |
| Oncorhynchus masou | cherry salmon (masou salmon) |
| Oncorhynchus nerka | Sockeye salmon |
| Oncorhynchus tshawytscha | (Chinook salmon) |
| Prosopium cylindraceum | Round whitefish |
| Oncorhynchus clarki | Cutthroat trout |
| Oncorhynchus mykiss | Rainbow trout |
| Salmo salar | Atlantic salmon |
| Salmo trutta | Brown trout |
| Salmo trutta X S. fontinalis | Tiger hybrid-trout |
| Salvelinus alpinus | Arctic charr |
| Salvelinus confluentus | Bull trout |
| Salvelinus fontinalis | Brook trout |
| Salvelinus leucomaenis | Japanese charr (white spotted charr) |
| Salvelinus malma | Dolly varden (Miyabe charr) |
| Salvelinus namaycush | Lake trout |
| Thymallus thymallus | Grayling |

Some Members of the Serranidae Family

| | |
|---|---|
| Centropristis ocyurus | Bank sea bass |
| Centropristis philadelphicus | Rock sea bass |
| Centropristis striata | Black sea bass |
| Diplectrum bivittatum | Dwarf sandperch |
| Diplectrum formosum | Sand perch |
| Epinephelus flavolimbatus | Yellowedge grouper |
| Epinephelus morio | Red grouper |
| Serranus phoebe | Tattler |
| Serranus tortugarum | Chalk bass |

Some Members of the Sparidae family

| | |
|---|---|
| Archosargus probatocephalus | Sheepshead |
| Archosargus rhomboidalis | Sea bream |
| Calamus penna | Sheepshead porgy |
| Lagodon rhomboides | Pinfish |
| Pagrus Major | Red Sea bream |
| Sparus aurata | Gilthead Sea bream |
| Stenotomus chrysops | Scup |

Some Members of the Cichlidae family

| | |
|---|---|
| Aequidens latifrons | Blue acara |
| Cichlisoma nigrofasciatum | Congo cichlid |
| Crenichichla sp. | Pike cichlid |
| Pterophyllum scalare | Angel fish |
| Tilapia mossambica | Mozambique mouth breeder |
| Oreochromis spp | Tilapia |
| Sarotherodon aurea | Golden Tilapia |

Some Members of the Centrarchidae family

| | |
|---|---|
| Ambloplites rupestris | Rock bass |
| Centrarchus macropterus | Flier |
| Elassoma evergladei | Everglades pigmy sunfish |
| Elassoma okefenokee | Okefenokee pigmy sunfish |
| Elassoma zonatum | Banded pigmy sunfish |
| Enneacanthus gloriosus | Bluespotted sunfish |
| Enneacanthus obesus | Banded sunfish |
| Lepomis auritus | Redbreast sunfish |
| Lepomis cyanellus | Green sunfish |
| Lepomis cyanellus X L. gibbosus | Green × pumpkinseed |
| Lepomis gibbosus | Pumpkinseed |
| Lepomis gulosus | Warmouth |
| Lepomis humilis | Orange-spotted sunfish |
| Lepomis macrochirus | Bluegill |
| Lepomis megalotis | Longear sunfish |
| Micropterus coosae | Shoal bass |
| Micropterus dolomieui | Smallmouth bass |
| Micropterus punctulatus | Spotted bass |
| Micropterus salmoides | Largemouth bass |
| Pomoxis annularis | White crappie |
| Pomoxis nigromaculatus | Black crappie |

For purposes of description of the present invention, it shall be understood that the term “subject” does not include humans, but includes each of the animal types and that unless specifically mentioned as an exception, description of an aspect of the invention with regard to one type of animal shall be understood to include the other types mentioned herein.

For purposes of the present invention, the term “food-producing” and “livestock” animals shall be understood to include all animals bred for (human) consumption as well as horses, etc. A non-limiting list of such animals include those of the avian, ruminant or bovine, ovine, porcine (pigs), etc. families, aquatic animals including fish such as trout or salmon, crustaceans such as shrimp, lobsters, crabs, etc. and other species raised or harvested for human consumption. Avian shall be understood to include, for example, poultry including chickens, turkeys, capons, geese, duck, etc. Bovine shall be understood to include, for example, cattle, beef, veal, etc. Ovine shall be understood to include, sheep, lamb, etc. Goats are also contemplated.

The methods described herein can also be used on companion animals or humans, if desired. For purposes of the present invention, the term “companion” animal shall be understood to include horses, cats (feline), dogs (canine), and rabbit species.

For purposes of the present invention “effective amount” shall be understood to mean an amount that achieves a desired clinical result, i.e. increase lean muscle deposition in animals and/or decrease in fat tissue. By “increase”, it is contemplated that there is a measurable and statistically significant gain in lean tissue accretion in animals treated with the methods described herein. By “decrease”, it is contemplated that there is a measurable and statistically significant reduction in adipose (fat) tissue in animals treated with the methods described herein. Depending upon the specific ALK5 receptor inhibitor administered, the amount and length of time such agents are administered, the increase observed is at least about 5%, with gains of from about 10% to about 15% or greater being preferred when such treatments are administered for time periods of at least about 60 days. The actual amounts will depend upon several factors known to those of ordinary skill, including the specific agent employed, the species being treated, the size of the animal, the tissues being measured, etc.

The present invention contemplates using not only those ALK5 receptor inhibitors mentioned in the foregoing patents and applications but also all known compounds having similar pharmacologic activity with respect to ALK5 receptor inhibition. In a particular embodiment, such compounds have chemical structures that are within the scope of Formula II.

Those skilled in the art will appreciate that for some of the compounds of the invention, one isomer will show greater pharmacological activity than other isomers. Polymorphs of the compounds of the invention and their salts and solvates are contemplated as also being part of this invention.

Compounds of the invention with an amino group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for purposes of the invention.

Certain compounds of the invention are acidic (e.g., those compounds which possess a carboxyl group). These compounds form pharmaceutically acceptable salts with inorganic and organic bases. Examples of such salts are the sodium, potassium, calcium, aluminum, gold and silver salts. Also included are salts formed with pharmaceutically acceptable amines such as ammonia, alkyl amines, hydroxyalkylamines, N-methylglucamine and the like.

As used herein, “solvate” means a molecular or ionic complex of molecules or ions of solvent with those of solute (for example, one or more compounds of Formula I, isomers of the compounds of Formula I, or prodrugs of the compounds of Formula I). Non-limiting examples of useful solvents include polar, protic solvents such as water and/or alcohols (for example methanol).

Prodrugs of the compounds of Formula I are contemplated as being part of this invention. As used herein, “prodrug” means compounds that are drug precursors which, following administration to a subject, as defined herein, release the drug in vivo via some chemical or physiological process (e.g., a prodrug on being brought to the physiological pH or through enzyme action is converted to the desired drug form).

The daily dose of the compositions of the present invention administered to the subject (i.e. livestock animal) can range from about 0.01 to about 100 mg/kg per day, is with amounts preferably ranging from about 0.05 to about 50 mg/kg/day, more preferably from about 0.5 to about 30 mg/kg/day and still more preferably ranging from about 1.0 mg/kg to about 20 mg/kg per day, given in a single dose or divided doses either in the form of a pharmaceutically acceptable dosage form or as part of a suitable animal feed or chow. The exact dose, however, is determined by the artisan and is dependent on the potency of the compound administered, the species of non-human animal the compound is administered to, as well as factors such as the age, weight, condition and response of the subject.

For administration of pharmaceutically acceptable salts of the above compounds, the weights indicated above refer to the weight of the acid equivalent or the base equivalent of the therapeutic compound derived from the salt.

The term therapeutically effective amounts means that amount of a therapeutic agent of the composition, such as an ALK5 receptor inhibitor, optionally in combination with other pharmacological or therapeutic agents described below, that will elicit a biological or medical response of a tissue, system, or subject that is being sought by the administrator (such as a researcher or veterinarian) which includes an increase in lean muscle tissue and/or decreases in fat tissue.

Also contemplated as part of the invention are combinations of the ALK5 receptor inhibitor and another therapeutic composition, compound, etc. As used herein, “combination therapy” or “therapeutic combination” means the simultaneous or sequential administration of two or more therapeutic agents, one of which is an ALK5 receptor inhibitor, etc. as well as other therapeutic agents known to have a favorable or synergistic effect on livestock performance parameters described herein. A non-limiting list of such agents include leptin or compounds that stimulate the signal transduction pathway triggered by leptin.

Such administration includes coadministration of these therapeutic agents in a substantially simultaneous manner, such as in a single pharmaceutically acceptable dosage form such as a tablet or capsule having a fixed ratio of active ingredients or in multiple, separate dosage forms for each therapeutic agent. Also, such administration includes use of each type of therapeutic agent in a sequential manner. In either case, the treatment using the combination therapy will provide beneficial effects in increasing the lean muscle/tissue content and/or reducing fat tissue of the subject animal. Also contemplated are livestock feeds, chows, foods, etc. for administration of the ALK5 receptor inhibitor compositions, either alone or in combination with other agents. A potential advantage of the combination therapy disclosed herein may be a reduction in the required amount of an individual therapeutic compound or the overall total amount of therapeutic compounds that are required to achieve the therapeutic effect. By using a combination of therapeutic agents, the side effects of the individual compounds can be reduced as compared to a monotherapy, which can improve compliance. Also, therapeutic agents can be selected to provide a broader range of complementary effects or complementary modes of action.

The compositions and treatments can be administered by any suitable means which produce contact of these compounds with the site of action in the body, for example in the muscle tissue of a subject. The daily dosage for the various compositions and therapeutic combinations described above can be administered to a subject in a single dose or in multiple subdoses, as desired. Sustained release dosages can also be used. Where the auxiliary (secondary) agent and ALK5 receptor inhibitor(s) are administered in separate dosages, the number of doses of each component given per day may not necessarily be the same, e.g., one component may have a greater duration of activity and will therefore need to be administered less frequently. Also useful are solid form preparations which are intended to be converted, shortly before use. The compounds of the invention may also be deliverable via other routes of administration, but preferably the compound is administered orally to the non-human animal.

The invention also relates to a kit in which one or more separate units containing the desired ALK 5 receptor inhibitor(s) is included. The kit will preferably include directions for the administration and use of each component. The kit form is particularly advantageous when the separate components must be administered in different dosage forms (e.g., oral and parenteral) or are administered at different dosage intervals.

In a still further aspect of the invention there is provided a method of producing meat, comprising administering an effective amount of an ALK 5 receptor inhibitor to an animal for a time sufficient to increase the muscle mass thereof, slaughtering the is animal and obtaining the meat from the animal.

Illustrating the invention is the following example which, however, is not to be considered as limiting the invention to their details. Unless otherwise indicated, all parts and percentages in the following example, as well as throughout the specification, are by weight.

### EXAMPLE

In order to show that the inhibition of ALK5 produces an increase in lean tissue, rats were dosed with 10 mg/kg of

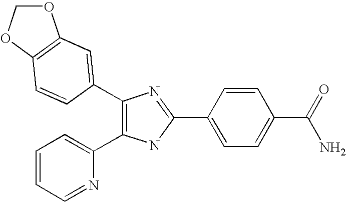

(hereinafter Compound A) in their feed for 39 days. Ten animals were treated with Compound A and a second group of ten animals served as a control group. Baseline values, in grams, of lean and fat tissue were assessed using Molecular Resonance Imaging technology for all animals. After 39 days of treatment, a highly significant increase in lean tissue of 19.6% (137.8 vs. 115.3, for the Compound A group vs. control group, respectively) was observed in the treated group compared to the control group (p<0.0036). See Table 1, below:

TABLE 1
THE EFFECT OF COMPOUND A ON LEAN AND
FAT TISSUE CONTENT IN RATS.
Results are expressed as the change in grams of tissue
from baseline values for the two treatment groups.

| | Fat (g) Δ | Lean (g) Δ |
|---|---|---|
| Control | | |
| 21 days | 52.1 | 82.0 |
| SE | 4.5 | 4.5 |
| 39 days | 77.9 | 115.3 |
| SE | 7.6 | 4.7 |
| Compound A @ 10 mg/kg | | |
| 21 days | 50.6 | 93.7 |
| SE | 3.0 | 3.8 |
| 39 days | 73.6 | 137.8** |
| SE | 4.8 | 4.8 |

**p - <0.0036

A separate observation was made in which a slight trend towards decreased fat content was also observed in those animals treated with inventive Compound A. These data support the invention that inhibition of ALK5 and/or ALK4 increases lean or muscle tissue through inhibition of the GDF-8 signaling pathway.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Therefore, we claim:

1. A method of increasing muscle tissue in animals, comprising administering an effective amount of an activin-like kinase (ALK) 5 receptor inhibitor to an animal.

2. The method of claim 1, wherein said ALK 5 receptor inhibitor is: 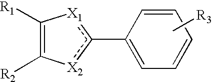
or a pharmaceutically acceptable salt or solvate thereof:
wherein:
R₁ is H, naphthyl or phenyl optionally substituted with one or more substituents selected from the group consisting of halo, —O—C₁₋₆ alkyl, —S—C₁₋₆alkyl, C₁₋₆ alkyl, C₁₋₆ haloalkyl, —O—(CH₂)ₙ₁—Ph, —S—(CH₂)ₙ₁-Ph, cyano, phenyl, and CO₂R₄, wherein R₄ is hydrogen or C₁₋₆ alkyl, and n1 is 0, 1, 2 or 3; or R₁ is phenyl fused with an aromatic or non-aromatic cyclic ring of 5-7 members wherein the cyclic ring optionally contains up to two heteroatoms, independently selected from N, O and S;
R₂ is H or 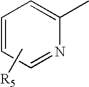
wherein R₅ is H, C-6 alkyl, C-6 alkoxy, phenyl, NH(CH₂)ₙ₂—Ph or NH—C₁₋₆alkyl, or halo, wherein n2 is 0, 1, 2 or 3;
R₃ is CONR₆R₇, CN, NO₂, C₁₋₆ alkylthio, —SO₂—, C₁₋₆ alkyl, C₁₋₆ alkoxy, SONH₂, CONHOH, NH₂, CHO, CH₂OH, CO₂R₆, tetrazole, OH, —S—C₁₋₁ alkyl, —SO—C₁₋₆alkyl, —O—C₁₋₁ alkyl, (CH₂)ₙ₃NH₂, CONHOR₆, O(CH₂)ₙ₃CO₂ R₆, O(CH₂)ₙ₃CONHR₆, CONHR₆, (CH₂)ₙ₃CO₂R₆, or (CH₂)ₙ₃CONHR₆, wherein Rr and R₇ are independently H or a C₁₋₆ alkyl and n3 is 0, 1, 2 or 3; and
one of X₁ and X₂ is N, S, O or CR₈, and the other is NR₈ or CHR₈, wherein R₈ is hydrogen, C₁₋₆ alkyl, or C₃₋₇ cycloalkyl, or when one of X₁ and X₂ is N or CR₈, then the other is S or O.

3. The method of claim 2, wherein said ALK 5 receptor inhibitor is: 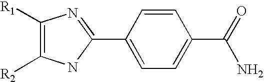
or a pharmaceutically acceptable salt or solvate thereof.

4. The method of claim 2, wherein said ALK 5 receptor inhibitor is 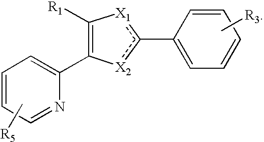
or a pharmaceutically acceptable salt or solvate thereof.

5. The method of claim 4, wherein R₁ is an optionally substituted naphthyl or phenyl.

6. The method of claim 4, wherein R₁ is phenyl optionally substituted with one or more substituents selected from the group consisting of halo, C₁₋₆ alkoxy, C₁₋₆ alkylthio, and phenyl; or R₁ is phenyl fused with an aromatic or non-aromatic cyclic ring of 5-7 members wherein the cyclic ring optionally contains up to two heteroatoms, independently selected from N, O and S, and is optionally substituted by ═O.

7. The method of claim 4, wherein R₃ is CO₂H, CONH₂, CN, CONHOH, CH₂OH, or tetrazole.

8. The method of claim 4, wherein one of X₁ and X₂ is N or CR₈, and the other is NR₈ or CHR₈, wherein R₈ is hydrogen, C₁₋₆ alkyl, or C₃₋₇ cycloalkyl, provided that at least one of X₁ and X₂ is N or NR₈; or one of X₁ and X₂ is N, and the other is O.

9. The method of claim 4, wherein one of X₁ and X₂ is N and the other is NR₈.

10. The method of claim 4, wherein each R₈ is hydrogen.

11. The method of claim 4, wherein said ALK5 receptor inhibitor is selected from the group consisting of:
4-[4-(4-Fluorophenyl)-5-(2-pyridyl)-1-hydroxy-1H-imidazol-2-yl]-benzonitrile;
4-[4-(4-Fluorophenyl)-5-(2-pyridyl)-1H-imidazol-2-yl]-benzonitrile;
4-[4-(4-Fluorophenyl)-5-(2-pyridyl)-1H-imidazol-2-yl]-benzoic acid;
Methyl 4-[4-(4-fluorophenyl)-5-(2-pyridyl)-1H-imidazol-2-yl]-benzoate;
Ethyl 4-[4-(4-fluorophenyl)-5-(2-pyridyl)-1H-imidazol-2-yl]-benzoate;
4-(4-Benzo[1,3]dioxol-5-yl-1-hydroxy-5-pyridin-2-yl-1H-imidazol-2-yl)-benzonitrile;
4-(4-Benzo[1,3]dioxol-5-yl-5-pyridin-2-yl-1H-imidazol-2-yl)-benzonitrile;
4-(4-Benzo[1,3]dioxol-5-yl-5-pyridin-2-yl-1H-imidazol-2-yl)-benzoic acid;
2-[4-Benzo[1,3]dioxol-5-yl-2-(4-nitrophenyl)-1H-imidazol-5-yl]-pyridine;
3-(4-Benzo[1,3]dioxol-5-yl-5-pyridin-2-yl-1H-imidazol-2-yl)-phenylamine;
4-[4-(4-Fluorophenyl)-2-(4-nitrophenyl)-1H-imidazol-5-yl]-pyridine;
4-[4-(4-Fluorophenyl)-5-pyridin-2-yl-1H-imidazol-2-yl]-phenylamine;
4-(4-Benzo[1,3]dioxol-5-yl-5-pyridin-2-yl-1H-imidazol-2-yl)-phenyl]methanol;
4-(4-Benzo[1,3]dioxol-5-yl-5-pyridin-2-yl-1H-imidazol-2-yl)-benzamide;
4-[4-(2,3-Dihydro-benzo[1,4]dioxin-6-yl)-5-pyridin-2-yl-1H-imidazol-2-yl]-benzonitrile;
4-[4-(2,3-Dihydro-benzo[1,4]dioxin-6-yl-5-pyridin-2-yl-1H-imidazol-2-yl]-benzamide;
4-[4-(2,3-Dihydro-benzofuran-5-yl)-5-pyridin-2-yl-1H-imidazol-2-yl]-benzamide;
3-[4-Benzo[1,3]dioxol-5-yl-5-pyridin-2-yl-1H-imidazol-2-yl]-benzonitrile;
4-[4-(2,3-Dihydro-benzofuran-6-yl)-5-pyridin-2-yl-1H-imidazol-2-yl]-benzonitrile;
4-[4-(2,3-Dihydro-benzofuran-6-yl)-5-pyridin-2-yl-1H-imidazol-2-yl]-benzamide;
3-(4-Benzo[1,3]dioxol-5-yl-5-pyridin-2-yl-1H-imidazol-2-yl)-benzoic acid;
4-[4-(4-Methoxyphenyl)-5-(2-pyridyl)-1H-imidazol-2yl]-benzonitrile;
4-[4-(2,2-Difluoro-benzo[1,3]dioxol-5-yl)-5-pyridin-2-yl-1H-imidazol-2-yl]-benzamide;
4-[4-(2,3-Dihydro-benzo[1,4]dioxin-6-yl)-1-methyl-5-pyridin-2-yl-1H-imidazol-2-yl]-benzamide;
4-[5-(2,3-Dihydro-benzo[1,4]dioxin-6-yl 1-methyl-4-pyridin-2-yl-1H-imidazol-2-yl]-benzamide;
4-(5-Benzo[1,3]dioxol-5-yl-4-pyridin-2-yl-oxazol-2-yl)benzonitrile;
4-(5-Benzo[1,3]dioxol-5-yl-4-pyridin-2-yl-oxazol-2-yl)-benzamide; and
4-(4-Benzo[1,3]dioxol-5-yl-5-pyridin-2-yl-1H-pyrrol-2-yl)-benzamide; or a
pharmaceutically acceptable salt or solvate thereof.

12. The method of claim 4, wherein said ALK5 receptor inhibitor is selected from the group consisting of:
4-(4-Benzo[1,3]dioxol-5-yl-5-pyridin-2-yl-1H-imidazol-2-yl)-phenol;
4-(4-Benzo[1,3]dioxol-5-yl-5-pyridin-2-yl-1H-imidazol-2-yl)-N-methy-1-benzamide;
4-(4-Benzo[1,3]dioxol-5-yl-5-pyridin-2-yl-1H-imidazol-2-yl)-N-methoxy-benzamide;
2-{4-Benzo[1,3]dioxol-5-yl-2-[4-(2H-tetrazol-5-yl)-phenyl]-1H-imidazol-5-yl}-pyridine;
[4-(4-Benzo[1,3]dioxol-5-yl-5-pyridin-2-yl-1H-imidazol-2-yl)-phenoxy]-acetic acid;
4-[4-(4-Fluoro-3-methoxyphenyl)-5-(6-methylpyridin-2-yl)-1H-imidazo-l-2-yl]-benzonitrile;
4-[4-(4-Fluoro-3-methoxyphenyl)-5-(6-methylpyridin-2-yl)-1H-imidazo-l-2-yl]-benzamide;
4-[4-(3-Fluoro-4-methoxyphenyl-5-(6-methylpyridin-2-yl)-1H-imidazo-l-2-yl]-benzonitrile;
4-[4-(3-Fluoro-4-methoxyphenyl-5-(6-methylpyridin-2-yl)-1H-imidaz-ol-2-yl]-benizamide;
4-[4-Benzo[1,2,5]oxadiazol-5-yl-5-(6-methyl-pyridin-2-yl)-1H-imidazol-2-yl]-benzonitrile;
4-[4-Benzo[1,2,5]oxadiazol-5-yl-5-(6-methylpyridin-2-yl)-1H-imidazo-l-2-yl]-benzamide;
4-[4-(6-Methoxynaphthalen-2-yl-5-(6-methylpyridin-2-yl)-1H-imidazo-l-2-yl]-benzonitrile;
4-[4-(6-Methoxynaphthalen-2-yl-5-(6-methylpyridin-2-yl)-1H-imidazo-l-2-yl]-benzamide;
4-[4-Benzo[1,2,5]thiadiazol-5-yl-5-(6-methylpyridin-2-yl)-1H-imidaz-ol-2-yl]-benzonitrile;
4-[4-Benzo[1,2,5]thiadiazol-5-yl-5-(6-methylpyridin-2-yl)-1H-imidaz-ol-2-yl]-benzamide
4-[4-Benzo[1,3]dioxol-5-yl-5-(6-methylpyridin-2-yl)-1H-imidazol-2-yl]-benzonitrile;
4-[4-Benzo[1,3]dioxol-5-yl-5-(6-methylpyridin-2-yl)-1H-imidazol-2-yl]-benzamide;
6-[2-(4-Cyanophenyl)-5-(6-methylpyridin-2-yl)-1H-imidazole-4-yl]-quinoxaline; and
6-[2-(4-Carboxamidophenyl)-5-(6-methylpyridin-2-yl)-1H-imidazole-4-yl]-quinoxaline;
and pharmaceutically acceptable salts or solvates thereof.

13. The method of claim 3, wherein said ALK 5 receptor inhibitor is

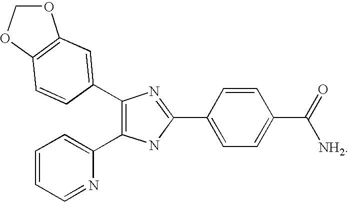

14. The method of claim 1, wherein said animal is selected from the group consisting of livestock, avian species, fish and swine.

15. The method of claim 1, wherein said animal is a livestock animal selected from the group consisting of cattle, poultry, pigs, goats and sheep.

16. The method of claim 14, wherein said avian species is selected from the group consisting of chickens, turkeys, ducks, geese and capons.

17. The method of claim 1, wherein the amount of ALK 5 inhibitor is from about 0.01 to about 100 mg/kg/day.

18. The method of claim 17, wherein the amount ALK 5 inhibitor is from about 0.05 to about 50 mg/kg/day.

19. The method of claim 18, wherein the amount ALK 5 inhibitor is from about 0.5 to about 30 mg/kg/day.

20. The method of claim 19, wherein the amount of ALK5 inhibitor is from about 1.0 to about 20 mg/kg/day.

21. The method of claim 1, wherein said administering of said ALK 5 receptor inhibitor results in a decrease in the amount of fat tissue in said animal.

22. The method of claim 1, wherein said administering of said ALK 5 receptor inhibitor results in an increase in the amount of lean tissue in said animal.

23. A livestock feed comprising an effective amount of: 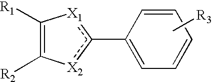
wherein:
R₁ is H, naphthyl or phenyl optionally substituted with one or more substituents selected from the group consisting of halo, —O—C₁₋₆ alkyl, —S—C₁₋₆ alkyl, C₁₋₆ alkyl, C₁₋₆ haloalkyl, —O—(CH₂)ₙ₁—Ph, —S—(CH₂)ₙ₁—Ph, cyano, phenyl, and CO₂R₄, wherein R₄ is hydrogen or C₁₋₆ alkyl, and n1 is 0, 1, 2 or 3; or R₁ is phenyl fused with an aromatic or non-aromatic cyclic ring of 5-7 members wherein the cyclic ring optionally contains up to two heteroatoms, independently selected from N, O and S;
R₂ is H or 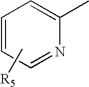
wherein R₅ is H, C₁₋₆ alkyl, C₁₋₆ alkoxy, phenyl, NH(CH₂)ₙ₂—Ph or NH—C₁₋₆ alkyl, or halo, wherein n2 is 0, 1, 2 or 3;
R₃ is CONR₆R₇, CN, NO₂, C₁₋₆ alkylthio, —SO₂—, C₁₋₆ alkyl, C₁₋₆ alkoxy, SONH₂, CONHOH, NH₂, CHO, CH₂OH, CO₂R₆, tetrazole, OH, —S—C₁₋₆ alkyl, —SO—C₁₋₆ alkyl, —O—C₁₋₆ alkyl, (CH₂)ₙ₃NH₂, CONHOR₆, O(CH₂)ₙ₃CO₂ R₆, O(CH₂)ₙ₃CONH R₆, CONH R₆, (CH₂)ₙ₃CO₂R₆, or (CH₂)ₙ₃CONHR₆, wherein R₆ and R₇ are independently H or a C₁₋₆ alkyl and n3 is 0, 1, 2 or 3; and
one of X₁ and X₂ is N, S, O or CR₈, and the other is NR₈ or CHR₈ wherein R₈ is hydrogen, C₁₋₆ alkyl, or C₃₋₇ cycloalkyl, or when one of X₁ and X₂ is N or CR₈, then the other is S or O.

24. A kit for increasing muscle deposition in animals, comprising an effective amount of: 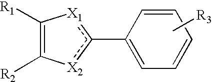
wherein:
R₁ is H, naphthyl or phenyl optionally substituted with one or more substituents selected from the group consisting of halo, —O—C₁₋₆ alkyl, —S—C₁₋₆ alkyl, C₁₋₆ alkyl, C₁₋₆ haloalkyl, —O—(CH₂)ₙ₁—Ph, —S—(CH₂)ₙ₁—Ph, cyano, phenyl, and CO₂R₄, wherein R₄ is hydrogen or C₁₋₆ alkyl, and n1 is 0, 1, 2 or 3; or R₁ is phenyl fused with an aromatic or non-aromatic cyclic ring of 5-7 members wherein the cyclic ring optionally contains up to two heteroatoms, independently selected from N, O and S;
R₂ is H or 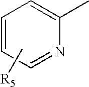
wherein R₅ is H, C₁₋₆ alkyl, C₁₋₆ alkoxy, phenyl, NH(CH₂)ₙ₂—Ph or NH—C₁₋₁₆ alkyl, or halo, wherein n2 is 0, 1, 2 or 3;
R₃ is CONR₆R₇, CN, NO₂, C₁₋₆ alkylthio, —SO₂—, C₁₋₄ alkyl, C₁₋₆ alkoxy, SONH₂, CONHOH, NH₂, CHO, CH₂OH, CO₂R₆, tetrazole, OH, —S—C₁₋₆ alkyl, —SO—C₁₋₆ alkyl, —O—C₁₋₆ alkyl, (CH₂)ₙ₃NH₂, CONHOR₆, O(CH₂)ₙ₃CO₂ R₆, O(CH₂)ₙ₃CONH R₆, CONH R₆, (CH₂)ₙ₃CO₂R₆, or (CH₂)ₙ₃CONHR₆, wherein R₆ and R₇ are independently H or a C₁₋₆ alkyl and n3 is 0, 1, 2 or 3; and
one of X₁ and X₂ is N, S, O or CR₈, and the other is NR₈ or CHR₈ wherein R₈ is hydrogen, C₁₋₆ alkyl, or C₃₋₇ cycloalkyl, or when one of X₁ and X₂ is N or CR₈, then the other is S or O.

25. A method of producing meat, comprising administering an effective amount of an ALK 5 receptor inhibitor to an animal for a time sufficient to increase the muscle mass thereof, slaughtering the animal and obtaining the meat from the animal.

26. A method of decreasing fat tissue in animals, comprising administering an effective amount of an activin-like kinase (ALK) 5 receptor inhibitor to an animal in need of such treatment.
